(19) United States
(12) Patent Application Publication
(10) Pub. No.: US 2007/0049568 A1
(43) Pub. Date: Mar. 1, 2007

(54) Control of induced elevated intraocular pressure

(75) Inventor: Paul Ashton, Boston, MA (US)

Correspondence Address: FISH & NEAVE IP GROUP;ROPES & GRAY LLP, ONE INTERNATIONAL PLACE, BOSTON, MA, 02110-2624, US

(73) Assignee: pSivida Inc., Watertown, MA (US)

(21) Appl. No.: 11/510,528

(22) Filed: Aug. 25, 2006

Related U.S. Application Data
(60) Provisional application No. 60/711,191, filed on Aug. 25, 2005.

(51) Int. Cl. A61K 31/57; A61K 31/138; A61K 31/56

(52) U.S. Cl. 514/170; 514/171; 514/651

(57) ABSTRACT

A variety of ocular diseases and conditions can be treated using corticosteroids or other compounds having glucocorticoid activity. A consequence of administration of such compounds is elevated intraocular pressure, which causes undesirable side-effects. The unwanted consequences of such elevated intraocular pressure may be prevented or treated through the administration of a steroid antagonist sufficient to counter the deleterious effect of the corticosteroid or other compound having glucocorticoid activity in the anterior chamber of the eye without countering the therapeutic effect of the treatment compound.

### CROSS REFERENCE TO RELATED APPLICATIONS

This application claims the benefit of U.S. Provisional Patent Application No. 60/711,191, filed on Aug. 25, 2005, the specification of which is hereby incorporated by reference in its entirety.

### BACKGROUND OF THE INVENTION

Corticosteroids and other compounds with glucocorticoid activity are known as effective therapeutic agents in the treatment of a wide variety of ocular conditions. Such compounds may be administered to the eye using a variety of local and systemic routes of delivery.

A drawback of ocular therapy involving corticosteroids and other compounds with glucocorticoid activity, however, is elevated intraocular pressure (IOP) resulting from the adverse effect of such compounds in the anterior chamber of the eye (in particular, in the trabecular meshwork). Elevation in IOP is of particular concern in patients who are already suffering from elevated IOP, such as glaucoma patients. Since therapy with corticosteroids and similar compounds is frequently long-term (i.e., several days or more), there is potential for significant damage to ocular tissue as a result of prolonged elevations in IOP attributable to that therapy.

What is needed is an effective method for the treatment or prevention of elevated IOP induced by corticosteroids or other compounds with glucocorticoid activity sufficient to counter the deleterious effect of such compounds in the anterior chamber of the eye without countering the therapeutic effect of the compound.

### SUMMARY OF THE INVENTION

One aspect of the invention is a method for the treatment or prevention of undesired side-effects induced by administration of a corticosteroid or other compound with glucocorticoid activity, comprising administering an amount of a steroid antagonist that is sufficient to counter a deleterious effect of such compound in the eye without substantially adversely affecting the therapeutic effect of the compound. In certain embodiments, the undesired side-effect is elevated intra-ocular pressure, cataract formation, scleral thinning and/or discoloration, or exophthalmos.

One aspect of the invention is a method for the treatment or prevention of elevated IOP induced by corticosteroids or other compounds with glucocorticoid activity, comprising administering an amount of a steroid antagonist that is sufficient to counter the deleterious effect of such compounds in the anterior chamber of the eye without adversely affecting the therapeutic effect of the compound. In certain embodiments, the steroid antagonist is selected from mifepristone (RU486), tetrahydrocortisol, bicalutamide, nilretamide, tamsulosin, and testolactone. In certain embodiments, the steroid antagonist is RU486.

Another aspect of the invention is a method for the treatment of an ocular disease or condition, comprising administering a therapeutically effective amount of a corticosteroid or other compound with glucocorticoid activity to treat the ocular disease or condition in combination with a steroid antagonist to counter the deleterious effects of the first compound in the anterior chamber of the eye. In certain embodiments, the steroid antagonist is RU486, tetrahydrocortisol, bicalutamide, nilretamide, tamsulosin, or testolactone. In certain embodiments, the steroid antagonist is RU486. Such conjoint treatment may be achieved by way of the simultaneous, sequential, or separate dosing of the individual components of the treatment. In certain embodiments, the corticosteroid or other compound with glucocorticoid activity and the steroid antagonist may together be in a single formulation.

### DETAILED DESCRIPTION OF THE INVENTION

The present invention provides a method for the treatment or prevention of undesired side-effects induced by a corticosteroid or other compound with glucocorticoid activity, comprising administering an amount of a steroid antagonist that is sufficient to counter a deleterious effect of such compound in the eye without substantially adversely affecting the therapeutic effect of the same compounds. In certain embodiments, the undesired side-effect is elevated intra-ocular pressure, cataract formation, scleral thinning and/or discoloration, or exophthalmos.

As used herein, the phrase “without substantially adversely affecting the therapeutic effect of the compound” and similar phrases mean that when a steroid antagonist is administered to a subject as described herein, the therapeutic effect of the corticosteroid or other compound with glucocorticoid activity is substantially the same as when the corticosteroid or other compound with glucocorticoid activity is administered in the absence of a steroid antagonist. The term “substantially the same activity” as used herein means that the therapeutic acitivity is at least about 80, about 85, about 90, about 95, or even about 99% of the activity level of the corticosteroid or other compound with glucocorticoid activity alone, e.g., the same therapeutic effect is seen as if the administered dose were 80%, 85%, 90%, 95% or even about 99% of the dose actually administered.

In certain embodiments, the invention relates to a method for the treatment of elevated intraocular pressure (IOP) induced by corticosteroids or other compounds with glucocorticoid activity, comprising administering an amount of a steroid antagonist, such as mifepristone (RU486), tetrahydrocortisol, bicalutamide, nilretamide, tamsulosin, or testolactone, sufficient to counter the deleterious effect of treatment compound in the anterior chamber of the eye without countering the therapeutic effect of the compound.

In certain embodiments, the invention relates to a method for the treatment of elevated IOP induced by a corticosteroid or other compound with glucocorticoid activity, comprising administering an amount of RU486 sufficient to counter a deleterious effect of treatment compound in the anterior chamber of the eye without substantially reducing the therapeutic effect of the compound.

The term “corticosteroids” as used herein, includes corticosteroids and other compounds having glucocorticoid activity.

Because corticosteroids can induce an increase in IOP, in certain embodiments this method may be employed in conjuction with the use of a corticosteroid for treating an ocular disease or condition, and thus comprises administering a therapeutically effective amount of a corticosteroid conjointly with a steroid antagonist, such as RU486, tetrahydrocortisol, bicalutamide, nilretamide, tamsulosin, or testolactone, preferably RU486, as described above. Similarly, the invention provides a method for preventing elevated IOP, comprising administering a therapeutically effective amount of a steroid antagonist, such as RU486, tetrahydrocortisol, bicalutamide, nilretamide, tamsulosin, or testolactone, preferably RU486. In certain such embodiments, the elevated IOP is an anticipated result of the administration of a corticosteroid compound for the treatment of an ocular condition or disease.

Normal eye pressure ranges from about 10 to about 21 mm Hg. Accordingly, as used herein, the terms “elevated IOP” or “increased IOP” refer to an eye pressure of greater than about 21 mmHg, or even greater than about 24 mm Hg.

In certain embodiments, the steroid antagonist is administered topically to the eye. In certain such embodiments, the steroid antagonist is administered in the form of eyedrops. In other embodiments, the steroid antagonist is administered subconjunctivally. In certain such embodiments, the steroid antagonist is administered via injection.

Suitable methods of administration of the steroid and/or steroid antagonist may also include rechargeable, biodegradable or nonerodible devices. Various slow release polymeric devices have been developed for the controlled delivery of drugs. A variety of biocompatible devices, some of which include polymers (including hydrogels), including both biodegradable and non-degradable polymers, can be used to form an implant for the sustained release of a drug at a particular target site. In certain embodiments, the steroid and/or steroid antagonist may be administered in a device described in U.S. Pat. No. 6,375,972, 6,217,895, or 6,548,078, and U.S. patent application Ser. No. 10/253,825, 10/714,677, 10/096,877, 10/428,214, 10/714,549 or 11/081,142, which are incorporated herein by reference in their entirety.

Suitable corticosteroids that may be used in the treatment of an ocular disease or condition include, but are not limited to, dexamethasone, fluorometholone, medrysone, betamethasone, triamcinolone, triamcinolone acetonide, prednisone, prednisolone, hydrocortisone, prednicarbate, deflazacort, halomethasone, tixocortol, prednylidene (21-diethylaminoacetate), prednival, paramethasone, methylprednisolone, meprednisone, mazipredone, isoflupredone, halopredone acetate, halcinonide, formocortal, flurandrenolide, fluprednisolone, flurprednidine acetate, fluperolone acetate, fluocortolone, fluocortin butyl, fluocinonide, fluocinolone acetonide, flunisolide, flumethasone, fludrocortisone, fluclorinide, enoxolone, difluprednate, diflucortolone, diflorasone diacetate, desoximetasone (desoxymethasone), desonide, descinolone, cortivazol, corticosterone, cortisone, cloprednol, clocortolone, clobetasone, clobetasol, chloroprednisone, cafestol, budesonide, beclomethasone, amcinonide, allopregnane acetonide, alclometasone, 21-acetoxypregnenolone, tralonide, diflorasone acetate, deacylcortivazol, RU-26988, budesonide, and deacylcortivazol oxetanone. All of the above-cited corticosteroids are known compounds. Further information about the compounds may be found, for example, in The Merck Index, Thirteenth Edition (2001), and the publications cited therein, the entire contents of which are hereby incorporated herein by reference. In certain embodiments, the corticosteroid is selected from fluocinolone acetonide, triamcinolone acetonide, and dexamethasone, or any combination thereof.

Other suitable compounds having glucocorticoid activity include, but are not limited to, soft steroids, such as loteprednol.

Ocular diseases or conditions that may be treated with steroids in combination with a steroid antagonist include, but are not limited to, macular dystrophy, ocular histoplamosis, presumed ocular histoplasmosis, glaucoma, proliferative vitreoretinopathy, macular edema, diabetic macular edema, age-related macular degeneration, diabetic retinopathy, uveitis, ocular neovascularization, diabetic retinopathy, retinal detachment, sickle cell retinopathy, retinal neovascularization, subretinal neovascularization, rubeosis, iritis, chronic posterior uveitis, uveitis affecting the posterior segment of the eye, pan uveitis, neoplasms, retinoblastoma, pseudoglioma, vascular diseases, retinal ischemia, choroidal vascular insufficiency, choroidal thrombosis, neovascularization of the optic nerve, cystoid macular edema, retinitis pigmentosa, retinal vein occlusion, angioid streak, retinal artery occlusion, and neovascularization due to penetration of the eye or ocular injury.

The precise time and frequency of administration and/or amount of the composition that will yield the most effective results in terms of efficacy of treatment in a given patient will depend upon the activity, pharmacokinetics, and bioavailability of a particular compound, physiological condition of the patient (including age, sex, disease type and stage, general physical condition, responsiveness to a given dosage, and type of medication), route of administration, etc. However, the above guidelines can be used as the basis for fine-tuning the treatment, e.g., determining the optimum time and/or amount of administration, which will require no more than routine experimentation, such as monitoring the subject and adjusting the dosage and/or timing.

In another aspect, the present invention provides pharmaceutical compositions. A composition for use in the subject method may be conveniently formulated for administration with a biologically acceptable medium, such as water, buffered saline, polyol (for example, glycerol, propylene glycol, liquid polyethylene glycol and the like) or suitable mixtures thereof. The optimum concentration of the active ingredient(s) in the chosen medium can be determined empirically, according to procedures well known to medicinal chemists. As used herein, “biologically acceptable medium” includes solvents, dispersion media, and the like which may be appropriate for the desired route of administration of the pharmaceutical preparation. Except insofar as any conventional media or agent is incompatible with treating vision impairment, its use in the pharmaceutical preparation of the invention is contemplated. Suitable vehicles and their formulation inclusive of other proteins are described, for example, in the book Remington's Pharmaceutical Sciences (Remington's Pharmaceutical Sciences. Mack Publishing Company, Easton, Pa., USA 1985).

Pharmaceutical formulations of the present invention can also include veterinary compositions, e.g., pharmaceutical preparations of a steroid antagonist suitable for veterinary uses, e.g., for the treatment of livestock or domestic animals, e.g., dogs.

Steroid antagonists of the present invention may be administered to the eye topically, such as by drops, lotions, solutions, ointments, gels or suspensions, or by injection, such as by subconjunctival or intraocular injection.

The agent(s) may be formulated with excipients such as methylcellulose, hydroxypropyl methylcellulose, hydroxypropyl cellulose, polyvinyl pyrrolidine, neutral poly(meth)acrylate esters, and other viscosity-enhancing agents. There are pharmaceutically acceptable excipients and additives customarily used for ophthalmic compositions known to the person skilled in the art, for example those of the type mentioned below, especially carriers, stabilizers, solubilizers, tonicity enhancing agents, buffer substances, anti-oxidants, preservatives, thickeners, complexing agents and other excipients. Examples of such additives and excipients can be found in U.S. Pat. Nos. 5,891,913, 5,134,124 and 4,906,613.

Formulations of the present invention in an embodiment are prepared, for example by mixing the active agent with the corresponding excipients and/or additives to form corresponding ophthalmic compositions. The active agent is preferably administered in the form of eye drops, the active agent being conventionally dissolved, for example, in a carrier. The solution is, where appropriate, adjusted and/or buffered to the desired pH and, where appropriate, a stabilizer, a solubilizer or a tonicity enhancing agent is added. Where appropriate, preservatives and/or other excipients are added to an ophthalmic formulation of the invention.

Carriers used in accordance with an embodiment of the present invention are typically suitable for topical or general administration, and are, for example, water, mixtures of water and water-miscible solvents, such as C₁₋₇alkanols, vegetable oils or mineral oils including from about 0.5% to about 5% by weight hydroxyethylcellulose, ethyl oleate, carboxymethylcellulose, polyvinylpyrrolidone and other non-toxic water-soluble polymers for ophthalmic uses, such as, for example, cellulose derivatives, such as methylcellulose, alkali metal salts of carboxymethylcellulose, hydroxymethylcellulose, hydroxyethylcellulose, methylhydroxypropylcellulose and hydroxypropylcellulose, acrylates or methacrylates, such as salts of polyacrylic acid or ethyl acrylate, polyacrylamides, natural products, such as gelatin, alginates, pectins, tragacanth, karaya gum, xanthan gum, carrageenin, agar and acacia, starch-derivatives, such as starch acetate and hydroxypropyl starch, and also other synthetic products, such as polyvinyl alcohol, polyvinylpyrrolidone, polyvinyl methyl ether, polyethylene oxide, preferably cross-linked polyacrylic acid, such as neutral Carbopol, or mixtures of those polymers. Preferred carriers include, for example, water, cellulose derivatives, such as methylcellulose, alkali metal salts of carboxymethylcellulose, hydroxymethylcellulose, hydroxyethylcellulose, methylhydroxypropylcellulose and hydroxypropylcellulose, neutral Carbopol, or mixtures thereof. The concentration of the carrier ranges, for example, from about 1 to about 100,000 times the concentration of the active ingredient.

The solubilizers used for an ophthalmic composition of the present invention in an embodiment include, for example, tyloxapol, fatty acid glycerol poly-lower alkylene glycol esters, fatty acid poly-lower alkylene glycol esters, polyethylene glycols, glycerol ethers vitamin E and vitamin E derivatives, such as Vitamin E Tocopherol Polyethylene Glycol 1000 Succinate (TPGS) or mixtures of those compounds. A specific example of a solubilizer is a reaction product of castor oil and ethylene oxide. Reaction products of castor oil and ethylene oxide have proved to be particularly good solubilizers that are tolerated extremely well by the eye. The concentration used depends especially on the concentration of the active ingredient. The amount added is typically sufficient to solubilize the active ingredient. For example, the concentration of the solubilizer ranges from about 0.1 to about 5000 times the concentration of the active ingredient pursuant to an embodiment of the present invention.

Examples of buffer substances are acetate, ascorbate, borate, hydrogen carbonate/carbonate, citrate, gluconate, lactate, phosphate, propionate, perborate TRIS (tromethamine) buffers and the like. Tromethamine and borate buffer are preferred buffers. The amount of buffer substance added is, for example, that necessary to ensure and maintain a physiologically tolerable pH range. The pH range is typically in the range of from about 5 to about 9, preferably from about 6 to about 8.2 and more preferably from about 6.8 to about 8.1.

Tonicity enhancing agents are, for example, ionic compounds, such as alkali metal or alkaline earth metal halides, such as, for example, CaCl₂, KBr, KCi, LiCl, NaI, NaBr or NaCl, or boric acid and the like. Non-ionic tonicity enhancing agents are, for example, urea, glycerol, sorbitol, mannitol, propylene glycol, dextrose and the like. For example, sufficient tonicity enhancing agent is added to impart to the ready-for-use ophthalmic composition an osmolality of approximately from about 50 mOsmol to about 1000 mOsmol, preferred from about 100 mOsmol to about 400 mOsmol, more preferred from about 200 mOsmol to about 400 mOsmol and even more preferred from about 280 mOsmol to about 350 mOsmol.

Examples of preservatives are quaternary ammonium salts, such as cetrimide, benzalkonium chloride or benzoxonium chloride, alkyl-mercury salts of thiosalicylic acid, such as, for example, thimerosal, phenylmercuric nitrate, phenylmercuric acetate or phenylmercuric borate, parabens, such as, for example, methylparaben or propylparaben, alcohols, such as, for example, chlorobutanol, benzyl alcohol or phenyl ethanol, guanidine derivatives, such as, for example, chlorohexidine or polyhexamethylene biguanide, or sorbic acid and the like. Where appropriate, a sufficient amount of preservative is added to the ophthalmic composition to ensure protection against secondary contaminations during use caused by bacteria and fungi.

Ophthalmic formulations of the present invention can also include, for example, non-toxic excipients, such as, for example, emulsifiers, wetting agents or fillers, such as, for example, the polyethylene glycols designated 200, 300, 400 and 600, or Carbowax designated 1000, 1500, 4000, 6000 and 10,000 and the like. Other excipients that may be used if desired are listed below but they are not intended to limit in any way the scope of the possible excipients. They include complexing agents, such as disodium-EDTA or EDTA; antioxidants, such as ascorbic acid, acetylcysteine, cysteine, sodium hydrogen sulfite, butyl-hydroxyanisole, butyl-hydroxytoluene or alphatocopherol acetate; stabilizers, such as a cyclodextrin, thiourea, thiosorbitol, sodium dioctyl sulfosuccinate or monothioglycerol vitamin E and vitamin E derivatives, such as Vitamin E Tocopherol Polyethylene Glycol 1000 Succinate (TPGS); or other excipients, such as, for example, lauric acid sorbitol ester, triethanol amine oleate or palmitic acid ester and the like. Preferred excipients are complexing agents, such as disodium-EDTA and stabilizers, such as a cyclodextrin and the like. Other preferred excipients include penetration enhancers such as benzalkonium chloride, Brij polymers such as PEG lauryl ether, and also dodecylmaltoside. The amount and type of excipient added is in accordance with the particular requirements and is generally in the range of from approximately 0.0001% by weight to approximately 90% by weight.

As indicated above a simple formulation of the present invention according to an embodiment includes an aqueous solvent which may be sterile water suitable for administration to the eye having an active agent dissolved, suspended or emulsified therein. However, preferred formulations of the present invention include the active agent dissolved in a formulation which is referred to in the art as an artificial tear formulation. Such artificial tear formulations are disclosed and described within U.S. Pat. Nos. 5,895,654, 5,627,611, and 5,591,426, as well as patents and publications cited and referred to in these patents, all of which are intended to be incorporated herein by reference.

Artificial tear formulations of the present invention in an embodiment promote good wettability and spread. Further, the artificial tear formulations preferably have good retention and stability on the eye and do not cause significant discomfort to the user. An exemplary artificial tear composition of the present invention includes:

(1) polyvinylpyrrolidone, preferably in the amount of about 0.1 to 5% by weight of said solution;

(2) benzalkonium chloride, preferably in an amount of about 0.01% to about 0.10% by weight;

(3) hydroxypropyl methylcellulose, preferably in an amount of about 0.2% to about 1.5% by weight of said solution; and

(4) glycerin, preferably in an amount of about 0.2% to about 1.0% by weight of said solution, wherein the composition is an aqueous solution having isotonic properties.

Those skilled in the art will recognize that a wide range of different formulations and artificial tear formulations can be utilized in connection with the present invention.

Additional ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatable with such fluids.

Formulations of the present invention can be administered in a manner generally known to those skilled in the art. In certain embodiments, the formulation is administered using an eyedropper. The eyedropper can be constructed in any suitable way.

It may be desirable to utilize a measured dose eyedropper of the type described within U.S. Pat. No. 5,514,118 or an illuminated eyedropper device of the type described in U.S. Pat. No. 5,584,823. A range of other eye droppers can also be utilized of the type described within the following U.S. Pat. Nos. 5,059,188; 4,834,727; 4,629,456; and 4,515,295. The patents cited here which disclose eyedroppers are incorporated herein by reference as are the various patents and publications cited and discussed within these patents.

Compositions usable for injection contain a physiologically tolerable carrier together with the relevant agent as described herein, dissolved or dispersed therein as an active ingredient. As used herein, the term “pharmaceutically acceptable” refers to compositions, carriers, diluents and reagents which represent materials that are capable of administration into, for example, the subconjunctival space without the production of undesirable physiological effects. The preparation of a injectable pharmacological composition typically contains active ingredients dissolved or dispersed therein. The preparation can also be emulsified. The active ingredient can be mixed with excipients which are pharmaceutically acceptable and compatible with the active ingredient and in amounts suitable for use in the therapeutic methods described herein. Suitable excipients are, for example, water, saline, sorbitol, glycerol or the like and combinations thereof. In addition, if desired, the composition can contain minor amounts of auxiliary substances such as wetting or emulsifying agents, pH buffering agents, and the like which enhance the effectiveness of the active ingredient. The composition can also contain viscosity enhancing agents like hyaluronic acid. The therapeutic composition of the present invention can include pharmaceutically acceptable salts of the components therein. Pharmaceutically acceptable salts include the acid addition salts that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

As used herein, the terms “prevent,” “prevention” and “preventing” are art-recognized, and when used in relation to a condition, such as a local recurrence (e.g., pain), a disease, a syndrome complex or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population. Prevention of pain includes, for example, reducing the magnitude of, or alternatively delaying, pain sensations experienced by subjects in a treated population versus an untreated control population.

As used herein, the terms “treat,” “treating” or “treatment” include reversing, reducing, or arresting the symptoms, clinical signs, and underlying pathology of a condition in manner to improve or stabilize a subject's condition.

From the foregoing description, one of ordinary skill in the art can easily ascertain the essential characteristics of the instant invention, and without departing from the spirit and scope thereof, can make various changes and/or modifications of the invention to adapt it to various usages and conditions. As such, these changes and/or modifications are properly, equitably and intended to be, within the full range of equivalence of the following claims.

All publications and patents mentioned herein are hereby incorporated by reference in their entirety, as if each individual publication or patent was specifically and individually indicated to be incorporated by reference.

We claim:

1. A method for the prevention of a deleterious effect induced by a corticosteroid or other compound having glucocorticoid activity, comprising administering a steroid antagonist in an amount sufficient to counteract a deleterious effect of the corticosteroid or other compound in the anterior chamber of the eye while maintaining a therapeutically effective level of the corticosteroid or other compound in the eye.

2. A method of claim 1, wherein the deleterious effect is selected from elevated intra-ocular pressure, cataract formation, scleral thinning, scleral discoloration, and exophthalmos.

3. A method of claim 2, wherein the deleterious effect is elevated intra-ocular pressure.

4. A method of claim 1, wherein the steroid antagonist is administered topically.

5. A method of claim 1, wherein the steroid antagonist is administered by injection.

6. A method of claim 1, wherein the steroid antagonist is administered in a sustained release formulation.

7. A method of claim 1, wherein the steroid antagonist is selected from RU486, tetrahydrocortisol, bicalutamide, nilretamide, tamsulosin, and testolactone.

8. A method of claim 7, wherein the steroid antagonist is RU486.

9. A method for the treatment of elevated intraocular pressure induced by corticosteroids or other compounds having glucocorticoid activity, comprising administering a steroid antagonist in an amount sufficient to counteract the deleterious effect of the corticosteroid or other compound in the anterior chamber of the eye while maintaining a therapeutically effective level of the corticosteroid or other compound in the eye.

10. A method of claim 9, wherein the steroid antagonist is administered topically.

11. A method of claim 9, wherein the steroid antagonist is administered by injection.

12. A method of claim 9, wherein the steroid antagonist is administered in a sustained release formulation.

13. A method of claim 9, wherein the steroid antagonist is RU486.

14. A method for treating an ocular disease or condition, comprising administering a corticosteroid or other compound having glucocorticoid activity conjointly with a steroid antagonist, wherein the steroid antagonist is administered in an amount sufficient to counteract the effect of the corticosteroid or other compound in the anterior chamber of the eye while maintaining a therapeutically effective level of the corticosteroid or other compound in the eye.

15. A method of claim 14, wherein the corticosteroid or other compound having glucocorticoid activity is administered via a sustained release delivery device.

16. A method of claim 14, wherein the steroid antagonist is administered topically.

17. A method of claim 14, wherein the steroid antagonist is administered by injection.

18. A method of claim 14, wherein the steroid antagonist is administered in a sustained release formulation.

19. A method of claim 14, wherein the steroid antagonist is selected from RU486, tetrahydrocortisol, bicalutamide, nilretamide, tamsulosin, and testolactone.

20. A method of claim 19, wherein the steroid antagonist is RU486.

21. A method of claim 14, wherein the corticosteroid is selected from dexamethasone, fluorometholone, medrysone, betamethasone, triamcinolone, triamcinolone acetonide, prednisone, prednisolone, hydrocortisone, prednicarbate, deflazacort, halomethasone, tixocortol, prednylidene (21-diethylaminoacetate), prednival, paramethasone, methylprednisolone, meprednisone, mazipredone, isoflupredone, halopredone acetate, halcinonide, formocortal, flurandrenolide, fluprednisolone, flurprednidine acetate, fluperolone acetate, fluocortolone, fluocortin butyl, fluocinonide, fluocinolone acetonide, flunisolide, flumethasone, fludrocortisone, fluclorinide, enoxolone, difluprednate, diflucortolone, diflorasone diacetate, desoximetasone (desoxymethasone), desonide, descinolone, cortivazol, corticosterone, cortisone, cloprednol, clocortolone, clobetasone, clobetasol, chloroprednisone, cafestol, budesonide, beclomethasone, amcinonide, allopregnane acetonide, alclometasone, 21-acetoxypregnenolone, tralonide, diflorasone acetate, deacylcortivazol, RU-26988, budesonide, deacylcortivazol oxetanone.

22. A method of claim 21, wherein the corticosteroid is selected from fluocinolone acetonide, triamcinolone acetonide, and dexamethasone.

23. A method of claim 14, wherein the compound is a soft steroid.

24. A method of claim 23, where in the soft steroid is loteprednol.

25. A method of claim 14, wherein the ocular disease or condition is selected from macular dystrophy, ocular histoplamosis, presumed ocular histoplasmosis, glaucoma, proliferative vitreoretinopathy, macular edema, diabetic macular edema, age-related macular degeneration, diabetic retinopathy, uveitis, ocular neovascularization, diabetic retinopathy, retinal detachment, sickle cell retinopathy, retinal neovascularization, subretinal neovascularization, rubeosis, iritis, chronic posterior uveitis, uveitis affecting the posterior segment of the eye, pan uveitis, neoplasms, retinoblastoma, pseudoglioma, vascular diseases, retinal ischemia, choroidal vascular insufficiency, choroidal thrombosis, neovascularization of the optic nerve, cystoid macular edema, retinitis pigmentosa, retinal vein occlusion, angioid streak, retinal artery occlusion, and neovascularization due to penetration of the eye or ocular injury.
